(19) United States
(12) Patent Application Publication
(10) Pub. No.: US 2022/0133507 A1
(43) Pub. Date: May 5, 2022

(54) AERATED EXTERNAL MAMMARY PROSTHESIS

(71) Applicant: Leonarda Sanchez, Bouloc (FR)

(72) Inventor: Leonarda Sanchez, Bouloc (FR)

(21) Appl. No.: 17/133,377

(22) PCT Filed: Jun. 27, 2019
(86) PCT No.: PCT/FR2019/051589

§ 371 Date: Aug. 30, 2021

(30) Foreign Application Priority Data
Jun. 27, 2018 (FR) 1855797

(51) Int. Cl. A61F 2/52

(52) U.S. Cl. CPC A61F 2/52; A61F 2002/526

(57) ABSTRACT

The invention relates to an external mammary prosthesis (1) comprising a framework (2) made of flexible material having the shape of a breast, said framework (2) defining an internal cavity and having, at a proximal end, an annular base (3) conformed to the shape of the chest of a person and, at a distal end, a solid disc zone (4) having the shape of a nipple and its adjacent areola, the disc zone (4) defining a sagittal axis, the annular base (3) being connected to the disc zone (4) by sinuous arms (5) made of flexible material, said arms (5) demarcating the internal cavity and being spaced apart from each other in order to define aeration zones (6) extending into the internal cavity.

### FIELD OF THE INVENTION/PRIOR ART

The invention relates to an external mammary prosthesis intended to replace a breast which has undergone a mastectomy, total or partial, and the appearance and dynamic behavior of which are similar to the natural breast.

### PRIOR ART

Depending on when the mastectomy operation took place, there are two types of prostheses.

The first type concerns so-called first-line transient mammary prostheses, which are intended to be worn immediately after the operation, and during the following two months, the time for healing. These prostheses are non-adherent: they do not attach directly to the skin, but are kept pressed against the chest by means of the bra inside which they are placed. These prostheses are conventionally made from textile. They more particularly take the form of a pad comprising a sleeve made from flexible textile material filled with a padding material. However, the prostheses known to date have two major drawbacks. First, the padding materials used, mainly cellulose, potentially comprise nanoparticles among which endocrine disruptors can be found. Secondly, when they are worn, the initial prostheses are pressed against the wounds from the operation, which prevents any ventilation of the latter during the entire period of wear and therefore slows their healing.

The second type concerns so-called second-line mammary prostheses, which are generally intended to be worn from the second month after the operation. These prostheses are conventionally made from silicone. They are designed as much as possible to have the same weight and appearance as the natural breast. In order to adjust the weight of the prosthesis to the user, it is known from application WO2015197938 to use ballast elements. While it is thus possible to obtain prostheses which are adjusted to replace breasts of a certain volume, it is difficult to provide suitable prostheses for replacing small breasts, the very structure of such a prosthesis proving to be too heavy from the outset. Also known from U.S. Pat. No. 401,028 is a prosthesis intended to be applied on the chest by simple pressure using a hand and held in position on the chest by suction effect when the pressure is released. The prosthesis in question consists of a non-perforated and elastic shell, of conical shape, and of a spring structure formed by twisted wires arranged on the surface of or inside the shell. The spring structure thus constitutes elastic reinforcing spacers allowing the deployment of the shell when the pressure applied to the latter is released. The drawback of such a prosthesis is its impermeability to air. Furthermore, the presence of the spring structure and its arrangement with the shell prevents any dynamic behavior of the prosthesis, which then offers a “fixed” appearance far removed from that of a natural breast.

The invention aims to remedy these problems by proposing an external mammary prosthesis making it possible to jointly overcome the drawbacks of the initial mammary prostheses and the second-line mammary prostheses.

Thus, the invention aims to provide an external mammary prosthesis devoid of nanoparticles capable of containing endocrine disruptors and ensuring aeration of the wounds so as to promote the healing of the wounds.

The invention also relates to a lightened external mammary prosthesis while exhibiting dynamic behavior close to a natural breast.

The invention also relates to a mammary prosthesis which can be worn equally well following a mastectomy, both total and partial.

The invention further relates to an external mammary prosthesis that can be used both as a first-line and a second-line prosthesis.

### SUBJECT MATTER OF THE INVENTION

To this end, and according to a first aspect, the invention provides an external mammary prosthesis comprising a framework of flexible material having the shape of a breast, said framework defining an internal cavity and having, at a proximal end, an annular base conformed to the shape of the chest of a person and, at a distal end, a solid disc zone having the shape of a nipple and its adjoining areola, the annular base being connected to the disc zone by sinuous arms made of flexible material, said arms demarcating the internal cavity and being spaced apart from each other in order to define aeration zones extending into the internal cavity.

By providing a framework which is thus open, air circulation is favored through the arms, thus avoiding keeping wounds in an environment in which secretions and moisture would stagnate, and promoting wound healing by allowing them to “breathe.” In addition, the sinuous shape of the arms allows the stresses to be distributed over their entire length, thus limiting the risk of the framework collapsing. The structure of the mammary prosthesis is also lightened. Finally, the existence of the internal space demarcated by the arms has the advantage of accommodating the remaining mass of the breast in the event of partial mastectomy, the mammary prosthesis thus filling the void left by the partial removal of the breast and offering a curve similar to that of the remaining breast.

Advantageously, the arms are distributed about the sagittal axis so as to have a spiral shape.

Advantageously, the arms are deformable jointly in rotation about the sagittal axis and in translation along this axis to allow the passage of the mammary prosthesis from a so-called usage position in which the arms are deployed to a so-called storage position in which the arms are folded. The expandable nature of the mammary prosthesis according to the invention thus makes it possible to ensure optimum bulk at the end of its storage.

Advantageously, when they are in the storage position, the arms and the disc zone are located in the same frontal plane.

Advantageously, the disc zone is housed, in the storage position, inside the annular base.

Advantageously, the annular base has a contact surface with the chest increasing toward the upper part of the mammary prosthesis.

Advantageously, the internal cavity is closed at the annular base by a cover having a face adapted to be pressed against the chest of the person.

Advantageously, the mammary prosthesis is formed in one piece.

### BRIEF DESCRIPTION OF THE FIGURES

Other objects and advantages of the invention will become apparent from the following description, given with reference to the appended drawings, in which:

FIG. 1 shows a schematic front view of an external mammary prosthesis according to the invention;

FIG. 2 shows a schematic rear view of the mammary prosthesis of FIG. 1;

FIG. 3 shows a schematic side view (right side) of the mammary prosthesis of FIG. 1;

FIG. 4 shows a schematic bottom view of the mammary prosthesis of FIG. 1;

FIG. 5 shows a schematic view of the mammary prosthesis in the storage position.

### DETAILED DESCRIPTION OF THE FIGURES

In the present description, the terms conventionally employed in the medical field of physiology are used to define the various spatial orientations relative to the body of a patient, in this case the frontal plane or the sagittal axis.

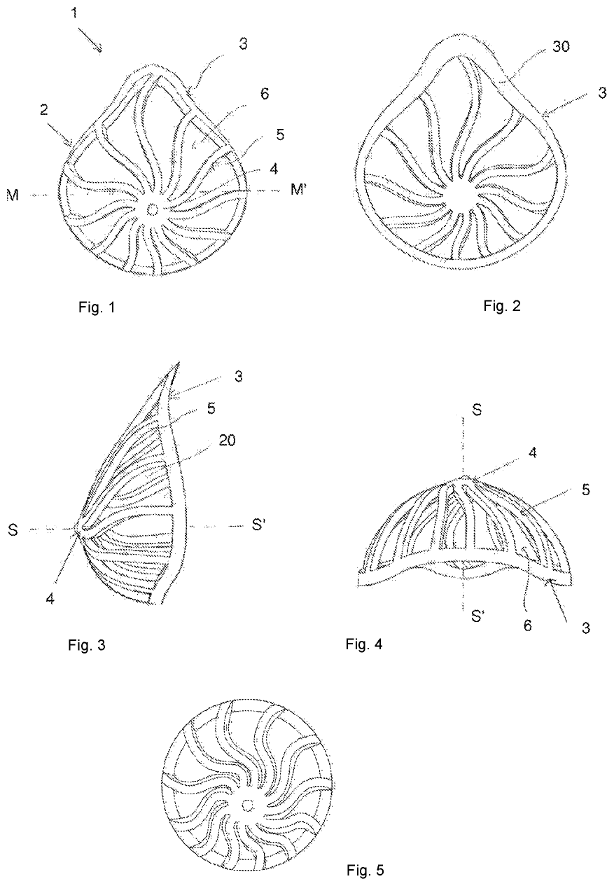

In relation to FIGS. 1 to 4, an embodiment is described of an external mammary prosthesis 1 according to the invention.

The mammary prosthesis 1 comprises a framework 2 made of flexible material having the shape of a breast. The framework 2 defines an internal cavity 20 having the shape of a spherical cap in the illustrated example. The framework 2 is preferably made of silicone.

The framework 2 according to the invention comprises, at the proximal end, an annular base 3 able to match the shape of the chest of a person and, at the distal end, a solid disc zone 4 having the shape of a nipple and its adjacent areola. The annular base 3 defines a space 30; the disc zone 4 defines a sagittal axis SS' perpendicular to the plane of the annular base 3.

The framework 2 further comprises arms 5 made of flexible material connecting the annular base 3 and the disc zone 4. The arms 5 form one and the same part with the disc zone 4 and the annular base 3. The arms 5 are arranged to define, with the directly adjacent arms, ventilation zones 6 opening into the internal cavity 20. The presence of such zones allows the passage of air and thus promotes healing when the mammary prosthesis 1 according to the invention is used in the context of a first-line prosthesis.

According to a preferred configuration of the invention, the arms 5 have a sinuous shape and are distributed about the sagittal axis SS' so as to have a spiral shape. In the illustrated embodiment, each arm 5 has an alternation of two radii of curvature. The radii of curvature of each arm can be the same or different. In order to give sufficient rigidity to the framework 2, the arms 5 are mounted between the annular base 3 and the disc zone 4 with a slight twist.

The framework 2 thus formed defines a prosthesis shell which better supports crushing from the weight of clothing with regard to the prostheses of the prior art on the one hand and ensures an optimal distribution of the weight and the pressure applied to the prosthesis on the other hand.

Due to their distribution around the distal zone and their flexible nature, the mammary prosthesis 1 is adapted to go from a so-called usage position in which the arms 5 are deployed to a so-called storage position in which the arms 5 are folded, as shown in FIG. 5. The passage from the usage position to the storage position of the mammary prosthesis 1 is obtained by bringing the arms 5 in a rotational movement about the sagittal axis SS' and by pressing at the disc zone 4 to bring the arms 5 in a translational movement along the same axis in a spatial direction 30. The passage from the storage position to the usage position of the prosthesis is in turn achieved by releasing the pressing force exerted at the disc zone 4, the arms 5 being deployed in a rotational movement in the inverse direction about the sagittal axis and a translational movement along this axis in the direction opposite the annular base 3. In the storage position, the arms 5 and the disc zone 4 are located in the same frontal plane, the disc zone 4 preferably being housed at the space 30 defined by the annular base 3 in order to optimize the size.

In order to ensure satisfactory contact with the users chest, the annular base 3 advantageously has a contact surface 30 with the chest which is larger in the upper part. The upper part of the mammary prosthesis is defined when it is worn. It is demarcated from the lower part by the median line MM′ illustrated in FIG. 1. FIG. 2 shows a contact surface 30 increasing toward the upper part of the mammary prosthesis 1. The advantage of providing a larger surface is to reinforce the strength of the framework 2.

Advantageously, the mammary prosthesis 1 comprises a cover (not shown) placed at the annular base 3 in order to close the internal cavity 20 demarcated by the framework 2. The cover has a face adapted to be pressed in whole or in part against the chest of the person. According to a particular configuration, the face of the cover is provided with impressions arranged to correspond to the shape and the volume of the wounds resulting from the mastectomy. The advantage of a structure thus arranged is that it allows any pressure on the scar to be reduced. Provision may also be made for the imprints to be made on an additional structure capable of being removably fixed to the cover.

The external mammary prosthesis described above is intended to be held in place against the chest using a bra, a brassiere or the like. The framework constitutes the prosthesis body as such and not a support structure for a shell or any additional element to the framework such as padding elements. In other words, the prosthesis is advantageously formed from the framework alone and the base of which is provided, where appropriate, with a cover as described above. Thanks to such an arrangement, the prosthesis according to the invention is “breathable.”

The invention is described in the foregoing by way of example. It is understood that a person skilled in the art is in a position to produce various variant embodiments of the invention without thereby departing from the scope of the invention.

1. External mammary prosthesis (1), characterized in that it comprises a framework (2) of flexible material having the shape of a breast, said framework (2) defining an internal cavity (20) and having, at a proximal end, an annular base (3) conformed to the shape of the chest of a person and, at a distal end, a solid disc zone (4) having the shape of a nipple and its adjoining areola, the disc zone (4) defining a sagittal axis, the annular base (3) being connected to the disc zone (4) by sinuous arms (5) made of flexible material, said arms (5) demarcating the internal cavity (20) and being spaced apart from each other in order to define aeration zones (6) extending into the internal cavity (20).

2. External mammary prosthesis (1) according to claim 1, characterized in that the arms (5) are distributed about the sagittal axis so as to have a spiral shape.

3. External mammary prosthesis (1) according to the preceding claim, characterized in that the arms (5) are deformable jointly in rotation about the sagittal axis and in translation along this axis to allow the passage of the mammary prosthesis (1) from a so-called usage position in which the arms (5) are deployed to a so-called storage position in which the arms (5) are folded.

4. External mammary prosthesis (1) according to claim 3, characterized in that in the storage position, the arms (5) and the disc zone (4) are located in the same frontal plane.

5. External mammary prosthesis (1) according to claim 3 or claim 4, characterized in that, in the storage position, the disc zone is housed inside the annular base (3).

6. External mammary prosthesis (1) according to one of the preceding claims, characterized in that the annular base (3) has a contact surface (30) with the chest increasing toward the upper part of the mammary prosthesis (1).

7. External mammary prosthesis (1) according to one of the preceding claims, characterized in that the internal cavity (20) is closed at the annular base (3) by a cover having a face adapted to be pressed against the chest of the person.

8. External mammary prosthesis (1) according to one of the preceding claims, characterized in that it is formed in one piece.
